(19) United States
(12) Patent Application Publication
(10) Pub. No.: US 2020/0139056 A1
(43) Pub. Date: May 7, 2020

(54) NOVEL SPRING-LOADED SUBCUTANEOUS AND INTERMUSCULAR INJECTOR DEVICE

(71) Applicant: Kyle REYNOLDS, Lewisville, TX (US)

(72) Inventor: Kyle REYNOLDS, Lewisville, TX (US)

(21) Appl. No.: 16/347,712

(22) PCT Filed: Jul. 26, 2017
(86) PCT No.: PCT/US17/43840

§ 371 Date: May 6, 2019

(51) Int. Cl. A61M 5/32; A61M 5/20; A61M 5/24

(52) U.S. Cl. CPC A61M 5/3298; A61M 5/2033; A61M 2005/2073; A61M 5/24; A61M 5/326

(57) ABSTRACT

The invention relates to a novel spring-loaded hypodermic injector device for subcutaneous and intramuscular injection. Specifically, the present invention incorporates a unique spring loaded, non-collinearly aligned double needle transfer/injector head. Also, when the injector is deployed a passive needle cover extends covering the needle and preventing an accidental strike of the needle after the injection is completed.

### CROSS-REFERENCE TO RELATED APPLICATIONS

Not applicable.

### FIELD OF THE INVENTION

The present invention relates to a novel spring-loaded hypodermic injector device for subcutaneous and intramuscular applications. Specifically, the present invention incorporates a unique spring loaded, non-collinearly aligned double needle transfer/injector head.

### BACKGROUND OF THE INVENTION

Currently, there is one accepted architecture for devices used to administer medications subcutaneously and intramuscularly by means of an auto-injector. Specifically, the EpiPen®, Auvi-Q®, Confidose®, Oval®, and all other auto-injectors are all linearly aligned injectors where the injection needle is collinearly aligned with the capsule of medication to be administered. This results in a comparatively long (five inches in the case of EpiPen®) injector system since the needle is lined up directly in front of the capsule of medication.

Moreover, given this physical architecture, the injector needle must be comparatively short and often does not achieve adequate intramuscular depth, particularly in heavier patients or in patients wearing heavier clothing.

Therefore, it is a first goal of the present invention to provide a simple and inexpensive auto-injector wherein the injection needle and the capsule of medication are not collinearly aligned.

It is a second goal of the present invention to provide a simple and inexpensive auto-injector wherein the injection needle and the capsule of medication are aligned along parallel axes.

It is a third goal of the present invention to provide a simple and inexpensive auto-injector wherein the length of the injection needle is such that it achieves deeper penetration depths.

It is a fourth goal of the present invention to provide a simple and inexpensive auto-injector wherein the injection needle is protected after use by a passive needle protector that covers the injection needle.

It is a fifth goal of the present invention to provide a simple and inexpensive auto-injector wherein the length of the injection needle is such that it may be used for subcutaneous through intramuscular injections.

Finally, it is a sixth goal of the present invention to provide a simple and inexpensive auto-injector that is relatively compact.

### SUMMARY OF THE INVENTION

The exemplary embodiment of the present invention comprises three general components: 1) An injector needle/transfer needle system with passive needle protector; 2) A transfer bladder; and, 3) A container of medication to be injected. These are all contained within a sealed carrying case with a deployment system.

The injector needle/transfer needle system comprises two hollow-core needles: a) A longer injection needle; and, b) A shorter transfer needle, fluidically connected together at their bases and extending parallel from a mounting bladder. The injector needle/transfer needle system has a retaining projection terminated by at least two retaining tangs. The at least two retaining tangs are generally conical in form. The injector needle/transfer needle system is powered, or deployed, by a first coiled or formed spring. The injector needle/transfer system needle also contains a slotted, concentric passive needle protector that emerges after the injector needle/transfer needle is withdrawn from the thigh. The passive needle protector protects the user from accidental needle strikes after the injection is completed and the needle is withdrawn. The passive needle protector is powered by a second coiled or formed spring.

The transfer bladder is a hollow bladder made of frangible, pierceable medical grade silicone or rubber. The transfer bladder has one centrally located preformed port circumferentially surrounded by a sealing zone where the top surface of the container of medication to be injected is seated. The preformed port leads to a hollow central lumen that extends the length of the transfer bladder.

The container of medication to be injected is a metal, plastic, or glass container containing the medication to be injected (epinephrine, insulin, etc.) with a mechanically operable injector and a third coiled or formed spring inserted into the open end.

The exemplary embodiment of the present invention is constructed in the following manner: First, the transfer bladder is inserted into the top of the carrying case with the sealing zone and central aperture facing out. Next, the container of medication is inserted such that its top surface is placed in the sealing zone of the transfer bladder so that the orifice at the top of the container of medication is located adjacent to the preformed port at the center of the sealing zone such that the medication in the container of medication may flow into the transfer bladder. Next, the injector needle/transfer needle system is placed in the carrying case such that the transfer needle is aligned with, though not in contact with, the transfer bladder and the injector needle with circumferential needle protector sleeve is aligned with and protrudes slightly from the exit port on the top surface of the carrying case. Next, the second coiled or formed spring associated with the passive needle protector is compressed and placed at the base of the passive needle protector sleeve. Next, the first coiled or formed spring associated with the injector needle/transfer needle system is compressed and placed at the base of the injector needle/transfer needle system. The injector needle/transfer needle system, the compressed first coiled or formed spring, the passive needle protector sleeve, and the compressed second coiled or formed spring are all held in place inside the carrying case by the retaining projection terminated by the at least two retaining tangs where the at least two retaining tangs are circumferentially restrained by a restraining aperture in a restraining partition. Just beneath the restraining partition is a release partition with a release aperture. The circumferential periphery of the release aperture just contacts the circumference of the at least two retaining tangs. Next, the third coiled or formed spring associated with the mechanical injector is compressed. Finally, the carrying case is closed and a sealing cap (if supplied) is place over the top.

The exemplary embodiment of the present invention is used in the following manner: First, the user removes the sealing cap (if supplied). Next, the user deploys the top surface of the carrying case such that the exposed end of the passive needle protector is against the thigh. The clothed epidermis covering the quadriceps femoris muscle on the front or side aspect of the thigh is ideally used. Next, the user presses the exposed end of the passive needle protector firmly against the thigh. Next, the passive needle protector is forced backwards against the injector needle/transfer needle which causes the injector needle/transfer needle system to be forced backwards causing the at least two retaining tangs to be radially compressed by the release aperture. This causes the retaining tangs under pressure from the first coiled or formed spring to rapidly pass through the retaining aperture and drive the injector needle/transfer needle system forward. Next, the transfer needle pierces and becomes fluidically coupled to the transfer bladder simultaneously with the injector needle penetrating through the clothing, the epidermis, the dermis, the hypodermis and into the quadriceps femoris muscle. Next, the medication inside the container of medication and the transfer bladder is propelled through the transfer needle, the mounting bladder, and the injection needle into the quadriceps femoris muscle under pressure cause by the third coiled or formed spring. Finally, the user removes the injection needle from the leg simultaneously causing the second coiled or formed spring to expand and deploy the slotted passive needle protector to be extended forward protecting the user from a subsequent strike of the injection needle tip.

### BRIEF DESCRIPTION OF THE DRAWINGS

FIG. 1 is a cutaway diagram showing the exemplary embodiment of the present invention in an undeployed state.

FIG. 2 is a cutaway diagram showing the exemplary embodiment of the present invention in a deployed state as the injection is occurring into the thigh.

FIG. 3 is a cutaway diagram showing the exemplary embodiment of the present invention in a deployed state after the injection has occurred and the needle has been removed from the thigh.

FIG. 4 is a modified cross-section showing the exemplary embodiment of the present invention.

### DETAILED DESCRIPTION OF THE PREFERRED EMBODIMENT

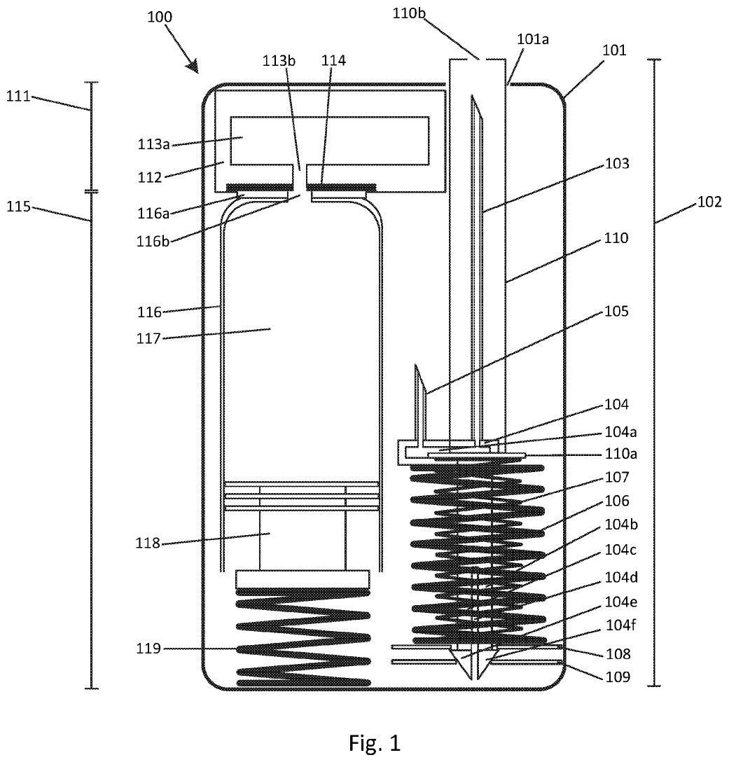

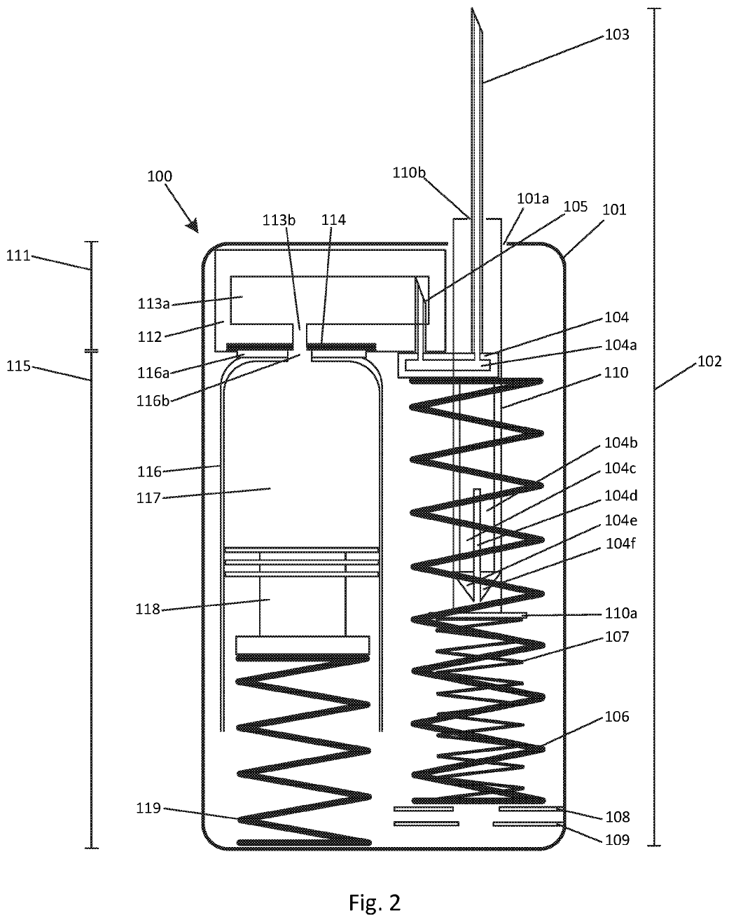

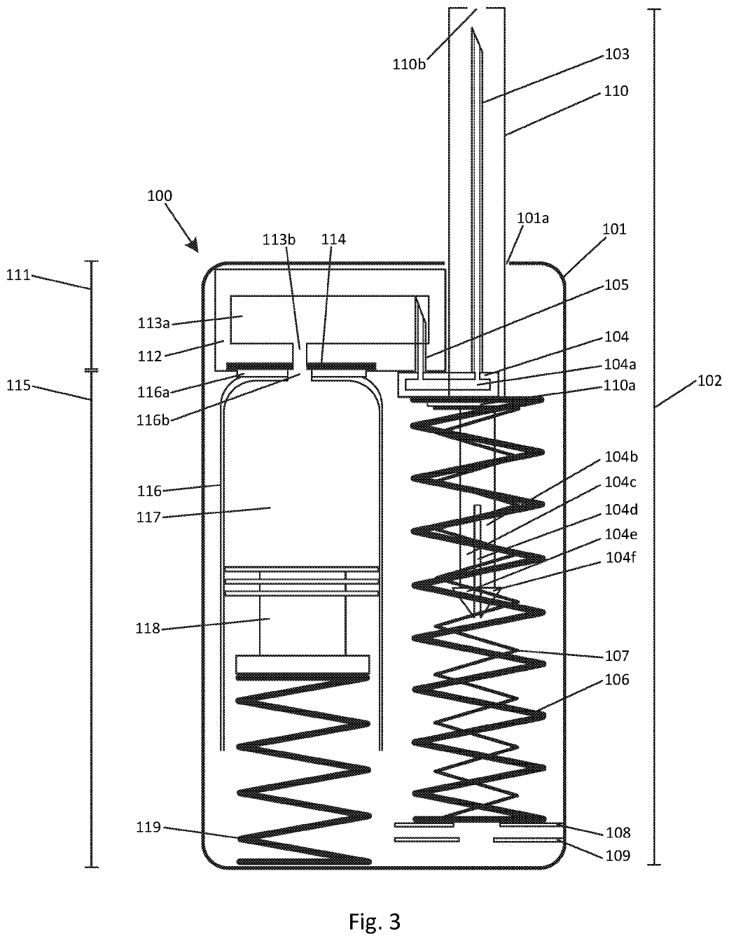

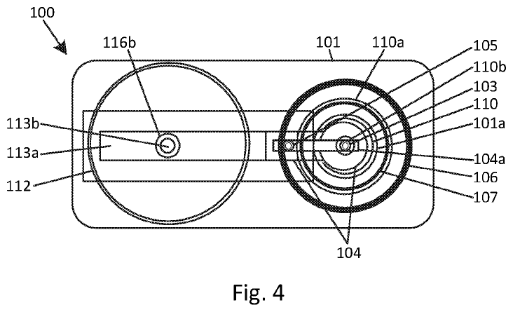

Turning now to FIGS. 1, 2, 3, and 4 an exemplary embodiment of the present invention 100 comprises the following general components: 1) An injector needle/transfer needle system 102; 2) A transfer bladder 111; and, 3) A container of medication 115 to be injected. These are all contained within sealed carrying case 101 retained inside by a restraining aperture in restraining partition 108. Just beneath the restraining partition is release partition 109 with a release aperture.

Injector needle/transfer needle system 102 comprises two hollow-core needles: a) Longer injection needle 103; and, b) Shorter transfer needle 105, fluidically connected together at their bases and extending parallel from mounting bladder 104. Mounting bladder 104 has a hollow mounting core 104a that fluidically connects the bases of injection needle 103 and transfer needle 105. Extending down from the base of mounting bladder 104 is a retaining projection divided into at least two retaining projection portions 104b and 104c. These at least two retaining projection portions 104b and 104c are terminated by retaining tangs 104e and 104f. Retaining tangs 104e and 104f are generally conical in form. Injector needle/transfer needle system 102 is powered, or deployed, by a first coiled or formed spring 106. Mounted circumferentially around injector needle/transfer needle system 102 is slotted, concentric passive needle protector 110 that emerges from carrying case 101 as part of the injector needle/transfer needle system 102 as injection needle 103 is withdrawn from the injection site. Passive needle protector 110 protects the user from accidental needle strikes after the injection is completed and injection needle 103 is withdrawn. Passive needle protector 110 is powered by second coiled or formed spring 107. Injector needle/transfer needle system 102 is powered by first coiled or formed spring 106 wrapped helically around, or beside, retaining projection portions 104b and 104c.

Injection needle 103 is about 22 gauge in diameter. Variants of injection needle 103 range in size from about 14 gauge to about 28 gauge. Injection needle 103 varies in length depending on the application: whether an adult or child is using the injector and whether an intramuscular or a subcutaneous injection is desired. For example, because of the injector architecture present in the prior art where the container of medication is collinearly aligned with the injector needle resulting in an injector that is relatively long, the length of the injector needle when fully deployed beyond the top of the injector is only about 0.5″ for children and about 0.63″ for adults. This is marginal for true intramuscular injections. Because the present invention allows for relatively longer lengths of injection needles 103, the length of injection needle 103 varies from about 0.5″ to about 1.5″ for intramuscular injections and from about 0.38″ to about 0.63″ for subcutaneous injections. These are the lengths of injection needle 103 measured extending from the top surface of passive needle protector 110 to the tip of injection needle 103 when injection needle 103 is fully deployed through central aperture 110b and passive needle protector 110 is fully retracted inside carrying case 101.

All three coiled or formed springs 106, 107, and 119 are constructed of stainless steel, high carbon steel, titanium, or other compounds such as Elgiloy®. For example, all three coiled or formed springs 106, 107, and 119 may, for example, be a Smalley crest-to-crest wave spring or a crest-to-crest wave spring with shim ends.

Further, first coiled or formed spring 106 has a compressed strength of about 4 pounds per inch to about 12 pounds per inch depending on the gauge of the needle. One embodiment of the present invention with a 22 gauge needle has a first coiled or formed spring 106 with a compressed strength of about 8 pounds per inch.

In an undeployed state, injector needle/transfer needle system 102 is locked on top of compressed first coiled or formed spring 106 wrapped helically around, or beside, retaining projection portions 104b and 104c. Injector needle/transfer needle system 102 is held in the locked, undeployed state by at least two retaining projection portions 104b and 104c which are terminated by retaining tangs 104e and 104f. Retaining tangs 104e and 104f are generally conical in form and when locked in an undeployed state they are expanded such that gap 104d is linear and parallel on its at least two internally facing sides. In its undeployed state, retaining tangs 104e and 104f are locked in the restraining aperture in restraining partition 108. In its undeployed state, the conical tips of retaining tangs 104e and 104f of pass partially through and abut a release aperture in release partition 109.

Transfer bladder 111 is comprised of a hollow bladder 112 made of frangible, pierceable medical grade silicone or rubber. Hollow bladder 112 has an open central lumen 113a accessed by one preformed port 113b circumferentially surrounded by sealing zone 114 where the top surface of container of medication 115 to be injected is seated and held. Sealing zone 114 is molded, or formed, in the bottom surface of hollow bladder 112. In one embodiment of the present invention, sealing zone 114 is comprised of a non-frangible ring, or gasket, sealed along the bottom surface of hollow bladder 112.

Container of medication 115 to be injected is a metal, plastic, or glass container 116 containing the medication to be injected (epinephrine, insulin, etc.) with an aperture 116b in its top. One embodiment of container of medication 115 has a mating ring 116a circumferentially formed around the top, mating surface of container 116. In this embodiment, mating ring 116a is insinuated in, or on, sealing zone 114 of hollow bladder 112.

In the preferred embodiment, sealing zone 114 acts with the top surface of container of medication 115 so that aperture 116b and preformed port 113b are coaxially aligned. By this means, medication 117 in container 116 is free to flow into the central lumen 113a of hollow bladder 112. Medication 117 in container 116 is forced into the central lumen 113a of hollow bladder 112 by pressure of moveable piston 118 powered by third coiled or formed spring 119.

The exemplary embodiment of the present invention is constructed in the following manner: First, carrying case 101 is opened and transfer bladder 111 is inserted into the top of carrying case 101. Next, container of medication 115 is inserted such that the surface with mating ring 116a with aperture 116b is coaxially aligned with sealing zone 114 with preformed port 113b in hollow bladder 112. Next, injector needle/transfer needle system 102 surmounted by slotted, concentric passive needle protector 110 is placed in carrying case 101 such that transfer needle 105 is aligned with, though not in contact with, transfer bladder 111 and injection needle 103 is aligned with central aperture 110b on the top surface of passive needle protector 110. Next, passive needle protector 110 is aligned with aperture 101a of carrying case 101. Next, second coiled or formed spring 107 associated with passive needle protector 110 is compressed and placed against base plate 110a of passive needle protector 110. Next, first coiled or formed spring 106 is placed circumferentially over second coiled or formed spring 107 and against the base of mounting bladder 104. Next, while transfer needle 105 is held so that it does not penetrate hollow bladder 112, both second coiled or formed spring 107 and first coiled or formed spring 106 are linearly compressed and sealed inside carrying case 101 by restraining partition 108. Next, release partition 109 is placed over restraining partition 108. Next, injector needle/transfer needle system 102 surmounted by slotted, concentric passive needle protector 110 is pressed backwards inside carrying case 101 until generally conical retaining tangs 104e and 104f are pressed through, and restrained in, a restraining aperture in restraining partition 108 and the tips of generally pyramidal retaining tangs 104e and 104f contact the edges of a release aperture in release partition 109. Next, third coiled or formed spring 119 is placed on the base of moveable piston 118. Next, the base of carrying case 101 is closed compressing third coiled or formed spring 119 forcing medication 117 into hollow bladder 112. Finally, a cap (if present) is placed over the end of carrying case 101 from which passive needle protector 110 emerges.

The exemplary embodiment of the present invention is used in the following manner: First, the user removes the cap (if present). Next, the user positions the top surface of carrying case 101 such that the exposed end of passive needle protector 110 is against the anterior or lateral aspect of the thigh. The clothed epidermis covering the quadriceps femoris muscle on the front or side aspect of the thigh is ideally used. Next, the user presses passive needle protector 110 into the skin. This linearly shifts generally pyramidal retaining tangs 104e and 104f downward into the release aperture of release partition 109. This radially compresses generally pyramidal retaining tangs 104e and 104f so that they are drawn through the restraining aperture of the restraining partition 108 by first and second coiled or formed springs 106 and 107. Next, injector needle/transfer needle system 102 is carried forward by first coiled or formed spring 106 associated with it such that transfer needle 105 pierces and becomes fluidically coupled with central lumen 113a of hollow bladder 112 of transfer bladder 111 simultaneously with injection needle 103 penetrating through the clothing, the epidermis, the dermis, the hypodermis and into the quadriceps femoris muscle of the user's thigh. Next, medication 117 inside container 116 and central lumen 113a of hollow bladder 112 of transfer bladder 111 is propelled out of central lumen 113a of hollow bladder 112 of transfer bladder 111, through transfer needle 105, through hollow mounting core 104a of mounting bladder 104, and though injection needle 103 by third coiled or formed spring 119 acting against moveable piston 118 and into the medial or lateral aspect of the quadriceps femoris muscle. Next, after several seconds the user removes injection needle 103 from the leg and second coiled or formed spring 107 expands forcing passive needle protector 110 through aperture 101a of carrying case 101 to cover and protect exposed injection needle 103. This prevents an accidental strike by exposed injection needle 103.

While the present invention has been described in what are thought to be the most useful and practical embodiments, it will be readily apparent to those having skill in the art that other variations may be readily conceived and created. For example, internal rearrangement of components is possible such as first coiled or formed spring 106 being interchanged with second coiled or formed spring 107. In other words, first coiled or formed spring 106 drives passive needle protector 110 while second coiled or formed spring 107 drives mounting bladder 104 and attached transfer needle 105 and injection needle 103. Also, all variants of the invention that are not equipped with passive needle protector 110, base plate 110a, or the spring that drives it are readily conceived of. All such of these variations are implicitly included in the spirit and scope of the present disclosure.

What is claimed is:

1. A spring-loaded hypodermic injector device comprising:
a) a medication container containing medication to be administered fluidically coupled to a pierceable, medical grade silicone or rubber transfer bladder;
b) a third coiled or formed spring driving a moveable piston inserted in said medication container and mechanically isolated at its bottom end;
c) an injector needle and a transfer needle fluidically coupled by means of a mounting bladder such that said injector needle and said transfer needle are aligned in the same direction and parallel to one another; and
d) a first coiled or formed spring aligned behind said mounting bladder and mechanically restrained at its bottom end;
e) such that when said transfer needle is driven forward by said first coiled or formed spring so that it is fluidically coupled with said transfer bladder said medication is driven from said medication container and said transfer bladder through said transfer needle, said mounting bladder, and said injector needle by said third coiled or formed spring.

2. A spring-loaded hypodermic injector device of claim 1 further comprising a slotted passive needle protector concentrically arranged around said injector needle and powered by a second coiled or formed spring mechanically attached at its bottom end.

3. A spring-loaded hypodermic injector device of claim 2 wherein the injected length of the injected needle ranges from about 0.5″ to about 1.5″ for intramuscular injections.

4. A spring-loaded hypodermic injector device of claim 2 wherein the injected length of the injected needle ranges from about 0.38″ to about 0.63″ for subcutaneous injections.

5. A spring-loaded hypodermic injector device of claim 2 wherein the first coiled or formed spring is a coiled spring.

6. A spring-loaded hypodermic injector device of claim 2 wherein the first coiled or formed spring is a crest-to-crest wave spring.

7. A spring-loaded hypodermic injector device of claim 2 wherein the first coiled or formed spring is a crest-to-crest wave spring with shim ends.

8. A spring-loaded hypodermic injector device of claim 2 wherein the first coiled or formed spring has an injection strength of about 8 pounds per inch.

9. A spring-loaded hypodermic injector device of claim 2 wherein the first coiled or formed spring has an injection strength ranging from about 4 pounds per inch to about 12 pounds per inch.

10. A spring-loaded hypodermic injector device of claim 2 wherein the first coiled or formed spring is constructed of stainless steel.

11. A spring-loaded hypodermic injector device of claim 2 wherein the first coiled or formed spring is constructed of high carbon steel.

12. A spring-loaded hypodermic injector device of claim 2 wherein the first coiled or formed spring is constructed of titanium.

13. A spring-loaded hypodermic injector device of claim 2 wherein the first coiled or formed spring is constructed of Elgiloy.

14. A spring-loaded hypodermic injector device of claim 2 wherein the injector needle is about 22 gauge in diameter.

15. A spring-loaded hypodermic injector device of claim 2 wherein the injector needle ranges from about 14 gauge to about 28 gauge in diameter.

16. A spring-loaded hypodermic injector device of claim 2 wherein the medication is epinephrine.

17. A spring-loaded hypodermic injector device of claim 2 wherein the medication is insulin.
